(19) United States
(12) Patent Application Publication
(10) Pub. No.: US 2015/0306324 A1
(43) Pub. Date: Oct. 29, 2015

(54) SELF-CONTAINED CONTINUOUS POSITIVE AIRWAY PRESSURE MASK AND METHOD OF USE

(71) Applicants: Arturo Alejo Ayon, San Antonio, TX (US); Joseph David Barrios, San Antonio, TX (US); Eliud Gutierrez, III, San Antonio, TX (US); Maxim Markichev Perkins, San Antonio, TX (US); Aaron Mosqueda, San Antonio, TX (US); Michael Paul Didion, San Antonio, TX (US)

(72) Inventors: Arturo Alejo Ayon, San Antonio, TX (US); Joseph David Barrios, San Antonio, TX (US); Eliud Gutierrez, III, San Antonio, TX (US); Maxim Markichev Perkins, San Antonio, TX (US); Aaron Mosqueda, San Antonio, TX (US); Michael Paul Didion, San Antonio, TX (US)

(21) Appl. No.: 14/700,112

(22) Filed: Apr. 29, 2015

Related U.S. Application Data
(60) Provisional application No. 61/985,531, filed on Apr. 29, 2014.

(51) Int. Cl. A61M 16/00; A61M 16/06

(52) U.S. Cl. CPC A61M 16/0066; A61M 16/0003; A61M 16/0633; A61M 2205/3344; A61M 2205/8206; A61M 2205/8262; A61M 2205/42

(57) ABSTRACT

The disclosed Current Continuous Positive Airway Pressure (CPAP) machines consist of a three piece system; mask, blower housing, and a house. The present invention discloses a self-contained CPAP mask that is configured to be fitted to a wearer's face and is comprised in an embodiment of a mask base component, a blower component, a silencing component, a blower cover component, a humidification component, and a control system component. This invention improves upon current CPAP devices by eliminating the hose and blower housing, allowing for increased mobility and portability.

### CROSS-REFERENCE TO RELATED APPLICATIONS

This application claims the benefit under Title 35 United States Code §119(e) of U.S. Provisional Patent Application Ser. No. 61/985,531; Filed: Apr. 29, 2014, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use directed to alleviating sleep apnea. More specifically, the present invention relates to a device and method of use for a continuous positive airway pressure (CPAP) mask.

### BACKGROUND OF THE INVENTION

Without limiting the scope of the disclosed device and method, the background is described in connection with a novel device and approach directed to a continuous positive airway pressure mask.

The word apnea comes from the Greek and means ‘without breath’. There are three different kinds of sleep apnea: obstructive, central, and mixed. Obstructive sleep apnea (OSA) is the most prevalent variety and is one of the types of sleep apnea the present invention is most heavily aimed at treating. OSA is caused when the airway is blocked by tissue during sleep, causing the person to wake up repeatedly throughout the night. The resulting lack of REM sleep, if left untreated, can cause sufferers of OSA to not only have poor daily performance, but potentially develop high blood pressure, heart problems, memory loss, impotency, weight gain, and headaches. OSA can also result in a loss of focus or drowsiness, which has been linked to accidents, injuries, and even death, both on and off the job. This has become such a severe concern the Department of Transportation is requiring all truck drivers to be checked for sleep apnea.

The most commonly prescribed treatment for OSA currently is a continuous positive air pressure (CPAP) machine. This device maintains a continuous positive air pressure on the mouth and/or nose. The purpose is to use the constant airflow to prevent any obstruction that may occur while sleeping. The current CPAP machines on the market consist of an AC power CPAP blower machine (usually sits on bedside table or a separate stand), a hose which connects to the mask, and a mask which is then secured to the head of the patient and sealed to the nose, mouth, or both, depending on the type of unit.

Today, 22 million Americans have already been diagnosed with mild to severe sleep apnea, while an estimated 103.5 million go undiagnosed. A Department of Transportation mandate, passed in 2013, requires all commercial licensed drivers to be checked for signs of sleep apnea to retain the ability to legally drive a commercial vehicle. This, along with the recent release of an affordable, at home sleep apnea test, which reduces testing costs by as much as 90% for the patient, projects the sleep apnea market to grow rapidly to a $19.72 billion industry by 2017.

Currently only about 40% of patients who are prescribed and own a CPAP machine actually use it as often as instructed. This means, despite the known health risks of non-compliance, thousands of dollars spent on the equipment, and a decreased quality of life 60% of users would prefer to not wear the CPAP machine because it is very uncomfortable. An object of the present invention aims to provide a solution that enables more patients to wear a CPAP machine.

While all of the aforementioned approaches may fulfill their unique purposes, none of them fulfill the need for a practical and effective means for providing a self-contained continuous positive airway pressure mask that is also quiet and comfortable.

The present invention therefore proposes a novel device and method of use for a self-contained continuous positive airway pressure mask that addresses the shortcomings of the prior art.

### BRIEF SUMMARY OF THE INVENTION

The present invention, therefore, provides a device and method of use for a self-contained continuous positive airway pressure mask.

In embodiments of the invention the self-contained CPAP mask is comprised of a mask base component, a blower component, a blower cover component, and a control system component. The mask base component is configured to be placed and secured to a wearer's face. In another embodiment, the mask is configured to be fitted to a patients face and is comprised of a centrifugal blower component, a silencing component, a control system component comprised of pressure sensors, a humidification component comprised of a humidification tray and wick, and a power supply. In other embodiments instead of a humidification wick, a humidification sponge is used. In embodiments the mask is configured with a power supply. In embodiments, this power supply is housed within the mask and in other embodiments is in a separate power supply housing connected by banded wires.

In some embodiments the invention is configured to provide a positive pressure range of 4-20 cm H₂O above atmospheric pressure. The mask may also supply humidified air via a humidification wick held by a humidification tray. The wick is an absorbent material that draws up water from the reservoir and provides a large surface area for it to evaporate from. The fan blows air over the wick to aid in the evaporation of the water. In some embodiments a pressure sensor located/mounted on the side or inside of the mask serves to control the blower output and maintain proper pressure supplied to the patient. In some embodiments the mask may be designed for use with already available components such as; facemask seal cushion, forehead pad, anti-asphyxiation pad, and head straps.

In some embodiments the humidification liquid is water. In some preferred embodiments it is distilled water. In yet other embodiments the humidification fluid may be a mixture of water and other fluids. In some embodiments that other fluid may be propylene glycol. In some embodiments the water to propylene glycol ratio is 1:1. In some embodiments the fluid may be normal saline solution.

Some embodiments of the invention include a noise reduction component. In some embodiments the noise reduction component is comprised of an upstream noise reduction component and a downstream noise reduction component that streamlines the air upstream and downstream of the blower resulting in reduced mechanical vibration and noise. The noise reduction components may be a separate entity that may be inserted or connected to the blower intake and exit. The noise reduction components may also be integrated into the mask as one whole entity. The embodiments may utilize a method to streamline the airflow through the device to avoid turbulent air contact with the impeller blades of the blower, and turbulent air exiting the blower. The embodiments may include the utilization of mechanical vibration absorbing materials, which may include foams, pastes, paints, layers, and seals. In some embodiments the material is Polydimethylsiloxane (PDMS), which is used at the contact interfaces of the mask components (blower cover, mask back, humidity tray) which reduces vibrations that would result in noise amplification. Sound deadening material is also to be included on the inside of the blower cover to reduce noise and vibration.

In some embodiments humidification is provided through use of a piezoelectric pump. The piezoelectric pump may be used in series with a water pouch, pumping water through an tube to the mask where the water is either dispersed through a diffuser nozzle directly inside of the mask or into a wick where the water can further evaporate avoiding condensation buildup. The water pouch may be located somewhere near the power supply housing. Using a piezoelectric micro-pump allows control of the supplied humidity to the patient. The currently available products have the ability to regulate flow and pressure output of the pump. With this known, the amount of mL/min of H₂O can be adjusted by matters of programming, giving control of the humidity supplied.

In some embodiments humidification is provided via damp sponge/wick housed in a tray in the mask, using the airflow over the wick to increase evaporation. In yet other embodiments humidity is to be supplied by a combination of a humidification wick and piezoelectric pump using the same setup as described previously, except instead of misting the water directly into the airflow the water is deposited into the humidification wick.

In embodiments, the control system component is comprised of the circuitry required for the system. In embodiments the control system component is mounted in the blower cover component. In some embodiments the control system component is further configured to use pressure sensor controls to control the speed of the blower using proportional-integral-derivative (PID) control. Other features of the housing may include a buzzer/speaker to warn the user when battery life is almost depleted, LCD screen, and a 4 button control (on/off, increase, decrease, enter). A rechargeable battery may also be located in the housing and powers the mask for continuous use. Data of a user may be recorded and saved to an SD card or other memory device for insurance purposes. The data logging included within the invention serves to ensure of user compliance and insurance purposes by recording pressure readings for the time duration of use. In other embodiments the control system may utilize a microprocessor, system-on-a-chip, FPGA, or ASIC. In yet other embodiments the control system may be partially or fully offloaded to an external computing device.

Also disclosed is a mask for treating sleep apnea comprising a self-contained CPAP mask configured to provide humidified air to a user wherein the mask can be placed and secured to a patients face and is comprised of a blower component, a silencing component, a control system component comprising a pressure sensor, and a humidification component comprising a humidification tray and wick. In some embodiments, the mask is further comprised of a power source or supply.

Other embodiments of the invention are discussed throughout this application. Any embodiment discussed with respect to one aspect applies to other aspects as well and vice versa. Each embodiment described herein is understood to be embodiments that are applicable to all aspects of the invention. It is contemplated that any embodiment discussed herein can be implemented with respect to any device, method, or composition, and vice versa. Furthermore, systems, compositions, and kits of the invention can be used to achieve methods of the invention.

In summary, the present invention generally relates to a device and method of use directed to alleviating sleep apnea. More specifically, the present invention relates to a device and method of use for a continuous positive airway pressure (CPAP) mask.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is an exploded view of the self-contained continuous positive airway pressure mask in accordance with embodiments of the disclosure;

FIG. 2 is a top a view of the downstream streamlining silencer component of the self-contained continuous positive airway pressure mask in accordance with embodiments of the disclosure;

FIG. 3 is a side view of the downstream streamlining silencer component of the self-contained continuous positive airway pressure mask in accordance with embodiments of the disclosure;

FIG. 4 is a perspective view of the downstream streamlining silencer component of the self-contained continuous positive airway pressure mask in accordance with embodiments of the disclosure;

FIG. 5 is a top view of the upstream streamlining silencer component of the self-contained continuous positive airway pressure mask in accordance with embodiments of the disclosure;

FIG. 6 is a side cross sectional view of the upstream streamlining silencer component of the self-contained continuous positive airway pressure mask in accordance with embodiments of the disclosure;

FIG. 7 is a perspective view of the upstream streamlining silencer component of the self-contained continuous positive airway pressure mask in accordance with embodiments of the disclosure;

FIG. 8 is a top view of the blower cover component of the self-contained continuous positive airway pressure mask in accordance with embodiments of the disclosure;

FIG. 9 is a front view of the blower cover component of the self-contained continuous positive airway pressure mask in accordance with embodiments of the disclosure;

FIG. 10 is a side view of the blower cover component of the self-contained continuous positive airway pressure mask in accordance with embodiments of the disclosure;

FIG. 11 is a perspective view of the blower cover component of the self-contained continuous positive airway pressure mask in accordance with embodiments of the disclosure;

FIG. 12 is a top view of the forehead assembly component of the self-contained continuous positive airway pressure mask in accordance with embodiments of the disclosure;

FIG. 13 is a side view of the forehead assembly component of the self-contained continuous positive airway pressure mask in accordance with embodiments of the disclosure;

FIG. 14 is a perspective view of the forehead assembly component of the self-contained continuous positive airway pressure mask in accordance with embodiments of the disclosure;

FIG. 15 is a front view of the forehead assembly component of the self-contained continuous positive airway pressure mask in accordance with embodiments of the disclosure;

FIG. 16 is a front view of the humidification tray component of the self-contained continuous positive airway pressure mask in accordance with embodiments of the disclosure;

FIG. 17 is a bottom view of the humidification tray component of the self-contained continuous positive airway pressure mask in accordance with embodiments of the disclosure;

FIG. 18 is a side view of the humidification tray component of the self-contained continuous positive airway pressure mask in accordance with embodiments of the disclosure;

FIG. 19 is a perspective view of the humidification tray component of the self-contained continuous positive airway pressure mask in accordance with embodiments of the disclosure;

FIG. 20 is a back view of the mask base component of the self-contained continuous positive airway pressure mask in accordance with embodiments of the disclosure;

FIG. 21 is a side view of the mask base component of the self-contained continuous positive airway pressure mask in accordance with embodiments of the disclosure;

FIG. 22 is a perspective view of the mask base component of the self-contained continuous positive airway pressure mask in accordance with embodiments of the disclosure;

FIG. 23 is a front view of the mask base component of the self-contained continuous positive airway pressure mask in accordance with embodiments of the disclosure;

FIG. 24 is a top view of the mask base component of the self-contained continuous positive airway pressure mask in accordance with embodiments of the disclosure;

FIG. 25 is a perspective view of the mask base component of the self-contained continuous positive airway pressure mask in accordance with embodiments of the disclosure;

FIG. 26 is a result set plot of relative humidity versus current of the self-contained continuous positive airway pressure mask in accordance with embodiments of the disclosure;

FIG. 27 is a result set plot of pressure versus current of the self-contained continuous positive airway pressure mask in accordance with embodiments of the disclosure;

FIG. 28 is an exploded view of the self-contained continuous positive airway pressure mask in accordance with embodiments of the disclosure;

FIG. 29 is a tip portion view of the intake component of the self-contained continuous positive airway pressure mask in accordance with embodiments of the disclosure;

FIG. 30 is a front view of the intake component of the self-contained continuous positive airway pressure mask in accordance with embodiments of the disclosure;

FIG. 31 is a perspective view of the intake component of the self-contained continuous positive airway pressure mask in accordance with embodiments of the disclosure;

FIG. 32 is a side view of the intake component of the self-contained continuous positive airway pressure mask in accordance with embodiments of the disclosure;

FIG. 33 is a top view of the mask base component of the self-contained continuous positive airway pressure mask in accordance with embodiments of the disclosure;

FIG. 34 is a front view of the mask base component of the self-contained continuous positive airway pressure mask in accordance with embodiments of the disclosure;

FIG. 35 is a side view of the mask base component of the self-contained continuous positive airway pressure mask in accordance with embodiments of the disclosure;

FIG. 36 is a perspective view of the mask base component of the self-contained continuous positive airway pressure mask in accordance with embodiments of the disclosure;

FIG. 37 is a bottom view of the silencing cover component of the self-contained continuous positive airway pressure mask in accordance with embodiments of the disclosure;

FIG. 38 is a perspective view of the silencing cover component of the self-contained continuous positive airway pressure mask in accordance with embodiments of the disclosure;

FIG. 39 is a front view of the silencing cover component of the self-contained continuous positive airway pressure mask in accordance with embodiments of the disclosure;

FIG. 40 is a side view of the silencing cover component of the self-contained continuous positive airway pressure mask in accordance with embodiments of the disclosure;

FIG. 41 is a side view of the diffuser component of the self-contained continuous positive airway pressure mask in accordance with embodiments of the disclosure;

FIG. 42 is a back view of the diffuser component of the self-contained continuous positive airway pressure mask in accordance with embodiments of the disclosure;

FIG. 43 is a perspective view of the diffuser component of the self-contained continuous positive airway pressure mask in accordance with embodiments of the disclosure;

FIG. 44 is a bottom view of the diffuser component of the self-contained continuous positive airway pressure mask in accordance with embodiments of the disclosure;

FIG. 45 is a top view of the blower cover component of the self-contained continuous positive airway pressure mask in accordance with embodiments of the disclosure;

FIG. 46 is a front view of the blower cover component of the self-contained continuous positive airway pressure mask in accordance with embodiments of the disclosure;

FIG. 47 is a side view of the blower cover component of the self-contained continuous positive airway pressure mask in accordance with embodiments of the disclosure;

FIG. 48 is a perspective view of the blower cover component of the self-contained continuous positive airway pressure mask in accordance with embodiments of the disclosure; and

FIG. 49 is a wiring schematic of the self-contained continuous positive airway pressure mask in accordance with embodiments of the disclosure.

### DETAILED DESCRIPTION OF THE INVENTION

Disclosed herein is an improved device and method of use for a self-contained continuous positive airway pressure mask. The numerous innovative teachings of the present invention will be described with particular reference to several embodiments (by way of example, and not of limitation).

The present invention is configured to offer patients the freedom and unit portability currently unknown to the CPAP market. By attaching the blower directly to the mask, eliminating the hose entirely, and attaching a battery pack to the mask itself instead of a tabletop unit, the invention addresses many of the pains and concerns of the current CPAP user.

In embodiments, the self-contained continuous positive airway pressure mask is comprised of a mask base component configured to be placed and secured to a wearer's face thereby forming a chamber between said base component and the wearer's face with an opening portion for receiving breathable gas; a blower component mounted to said mask base component, configured to create a pressure in said chamber; a blower cover component mounted to said mask base component and configured to cover the blower component; and a control system component for maintaining said pressure in said chamber.

Various embodiments of the invention may be configured where the blower component is for example and not a limitation, a S6 CPAP impeller from ResMed Limited. Various blowers may be used, such as axial fans, radial fans, centrifugal fans, etc. or any new technology able to deliver the required flow of air. The mask mask may be secured to a wearer's face by many means including straps.

The invention's mask in embodiments is a full-faced design that covers both the nose and mouth. In other embodiments, the mask only covers the nasal area or the oral region. In embodiments, the mask has the blower motor attached directly to the mask, and uses fluid-dynamics by streamlining the air entering and exiting the blower component. The streamlining of the air reduces turbulence which reduces noise. Having all components of the invention attached and integrated as part of the mask releases the user from their night table or unit stand, allowing a much larger range of motion for the wearer during the night. It is powered by a battery pack attached to the patient or to the mask itself, thus allowing it to move with the patient.

An embodiment of the invention provides 8-10 hours of user time and a pressure range of 2-20 cm H₂O. The sound level obtained is equal to or below 40 dbA. Some embodiments of the invention include an air filtration component to filter particles of approximately 3 μm in size (common dust particles). In some embodiments the filter is easy to remove and replace.

In some embodiments a ramp up time of 5 to 45 min in 5 min increments until preset pressure is reached may be incorporated to ease the wearer's transition to sleep and is implemented by an algorithm as part of the control system component that uses the pressure sensor to control the output of the blower. A flow range of 40-120 L/min is to be attained.

The integrated mask and air pump system provides positive air pressure for the treatment of sleep apnea as well as humidified air via a humidification component which in embodiments is a humidification wick. An incorporated air filtration system is used to filter common dust particles, typically ¼ inch in thickness and ranging from 0.5 to 3 inches in diameter. In embodiments, the pressure sensor is a diaphragm piezoelectric sensor with a gel coating specifically made for measurements in moist environments and low pressure readings as well as temperature readings. The pressure sensor is located within or as part of the mask and provides feedback as part of the control system to regulate the pressure based on the pump current or rpms. Temperatures for which humidification is to be beneficial are 70-80° F. Temperatures can be shown on the LCD screen via feedback from the pressure/temperature sensor.

In embodiments, the power supply component may be worn by the patient using an arm strap, waistband, or a holding mechanism for mounting to a headboard or wall. The power supply is compatible with a charger similar to that of a cell phone charger and compatible with a 120 V wall outlet. The housing supplies the necessary voltage (via rechargeable Li ion battery or an equivalent) to power the mask. In some embodiments an incorporation of LED lights or noise buzzer serves to notify the patient when battery life is insufficient. The power supply may receive power from any suitable power source, e.g., a wall power outlet, wall mounted transformers, a battery pack or other power storage medium. The power supply may be connected to the power source or the mask via a power cord. In one embodiment, sensors able to register and/or adjust to data received from sensors that may be provided in the mask (e.g. CO2, O2, humidity, pressure, flow, and/or temperature sensors). In one embodiment, the monitoring of sensors occurs via infrared technology or radio waves. A control box may be provided to adjust, e.g., the motor speed, e.g., for bi-level treatment, or other parameters relative to the information received through the sensors. Other embodiments may be to sense leak and adjust motor speed and thus delivery pressure or flow accordingly.

In some embodiments the sensors and control system may be interfaced with a portable computing device via wire or some form of wireless such as wi-fi or bluetooth. In some embodiments the portable computing device is a smartphone. In some embodiments the portable computing device can be used to set various parameters for the mask operation, or to provide an alert in the case of a malfunction or upon detection of an asphyxiation event. In some embodiments an alert can be auditory, visual, or via internet notification such as email or text message.

An increase/decrease controls such as buttons may be integrated into the mask to allow for control of different angular speeds for the blower impeller. An anti-asphyxiation valve is included in some embodiments to prevent suffocation upon machine failure. Upon machine failure the flow provided by the blower is absent and the valve is opened allowing the patient to maintain breathing. A forehead support component may be used to alleviate pressure typically felt at the bridge of the nose.

Several methods have been incorporated into the invention to assist in noise reduction and damping of mechanical vibrations. In one embodiment, the inside of the blower cover may be insulated with a thin layer of sound deadening material. These materials may be sound dampers, barriers, and/or absorbers More specifically, some materials may include but not be limited to open-cell urethane foams, fiberglass, various viscoelastic materials such as PMDS, and various elastomeric materials.

The intake and exit ports of the blower in embodiments are modified with silencer components in which the air at the intake and exit are now streamlined. Streamlining the air reduces turbulence which is responsible for noise. The base of the mask and all component contact interfaces may be coated with Polydimethylsiloxane (PDMS), rubber gaskets, or any other polymer based material to prevent air leaks and plastic-plastic contact. The PDMS also serves to hold the blower in its place using its semi-adhesive properties. Each of these aspects of the invention assists in noise and mechanical vibration reduction.

In embodiments of the mask, Table 1 and Table 2 reflect the performance values achieved.

TABLE 1

| current | voltage | % Relative Humidity |
|---|---|---|
| 0 | 12 | 46.7 |
| 0.2 | 12 | 47.8 |
| 0.25 | 12 | 48.3 |
| 0.3 | 12 | 49.1 |
| 0.35 | 12 | 49.9 |
| 0.4 | 12 | 50.4 |
| 0.45 | 12 | 51.2 |
| 0.5 | 12 | 51.9 |
| 0.55 | 12 | 52.4 |
| 0.6 | 12 | 52.6 |
| 0.65 | 12 | 52.8 |
| 0.7 | 12 | 52.9 |
| 0.75 | 12 | 52.7 |
| 0.8 | 12 | 52.4 |
| 0.85 | 12 | 52.1 |
| 0.9 | 12 | 51.7 |
| 0.96 | 12 | 51.3 |
| 1.05 | 12 | 50.9 |
| 1.1 | 12 | 50.5 |
| 1.15 | 12 | 50.2 |

TABLE 2

| current | voltage | pressure (in H2O) | pressure (cm H20) |
|---|---|---|---|
| 0.2 | 12 | 0 | 0 |
| 0.25 | 12 | 0.75 | 1.905 |
| 0.3 | 12 | 1.75 | 4.445 |
| 0.35 | 12 | 2.2 | 5.588 |
| 0.4 | 12 | 3 | 7.62 |
| 0.45 | 12 | 3.5 | 8.89 |
| 0.5 | 12 | 4.25 | 10.795 |
| 0.55 | 12 | 5 | 12.7 |
| 0.6 | 12 | 5.2 | 13.208 |
| 0.65 | 12 | 5.8 | 14.732 |
| 0.7 | 12 | 6.2 | 15.748 |
| 0.75 | 12 | 6.8 | 17.272 |
| 0.8 | 12 | 7.2 | 18.288 |
| 0.85 | 12 | 7.8 | 19.812 |
| 0.9 | 12 | 8.4 | 21.336 |
| 0.95 | 12 | 8.8 | 22.352 |
| 1 | 12 | 9 | 22.86 |
| 1.05 | 12 | 9.8 | 24.892 |
| 1.15 | 12 | 10.4 | 26.416 |

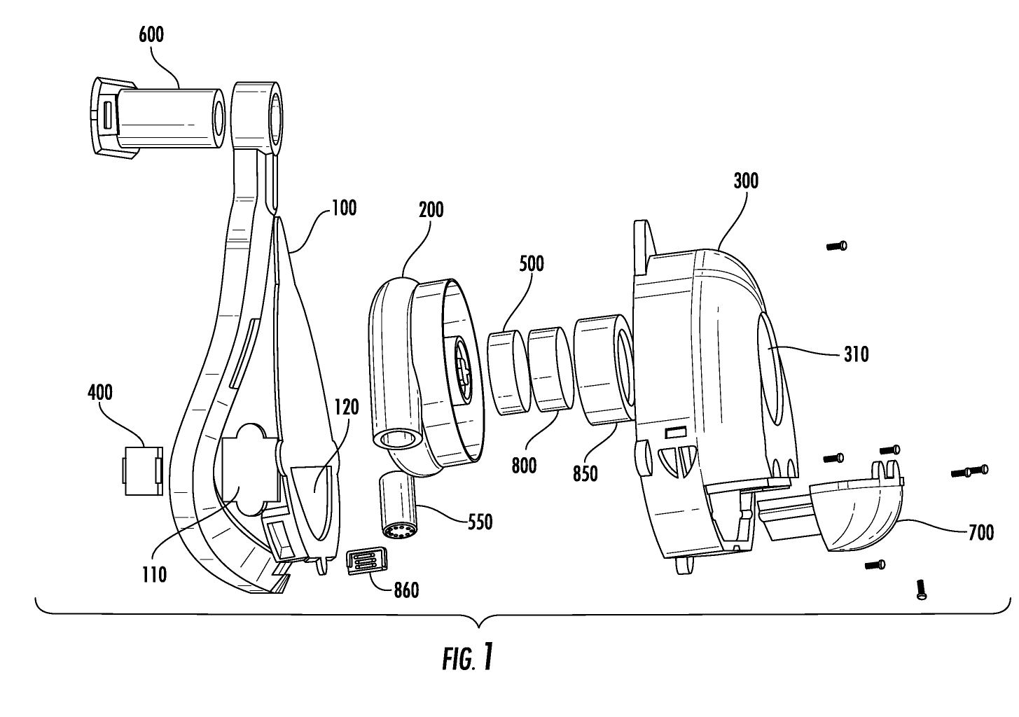

FIG. 1 is an exploded view of the self-contained continuous positive airway pressure mask in accordance with embodiments of the disclosure. Component 600 is the forehead assembly component used to alleviate weight felt on the bridge of the nose. Component 100 is the mask base component used to hold the major components of the mask such as the control system components (pressure sensor) 400 and the blower component 200. The pressure sensor 400 measures pressure and temperature on the inside of the mask and serves to control the blower component 200 output. The pressure sensor slot 110 may vary with respected to the pressure sensor chosen. The anti-asphyxiation polymer 860 is to prevent patient suffocation in the case of mask failure. The downstream streamline noise reducer (silencer) 550 streamlines the air exiting the blower component and avoids turbulence. The blower 200 is used to provide the positive pressure required to treat sleep apnea. The upstream streamline noise reducer (silencer) 500 which streamlines the air entering the blower making the intake of the blower component 200 more quiet. The air filter 800 filters out common dust particles from entering the blower component 200 and the wearer's airway. The filter cover 850 in an embodiment is threaded to attach to the intake silencer 500 and serves to hold the filter 800 in its proper location. The blower cover component 300 encloses the blower component 200 and control system components and in embodiments holds the humidification component 700 (in embodiments the sliding humidity tray) and air filter component 800 as well as the air filter cover component 850. The blower cover component in embodiments is mounted to the mask base component 100. In embodiments, the humidity tray 700 holds a humidification wick that is pre-saturated with water. In embodiments, the blower the anti-asphyxiation slot/valve that holds the anti-asphyxiation polymer in the proper position to where the valves/slot remains closed when the mask is on and open when off.

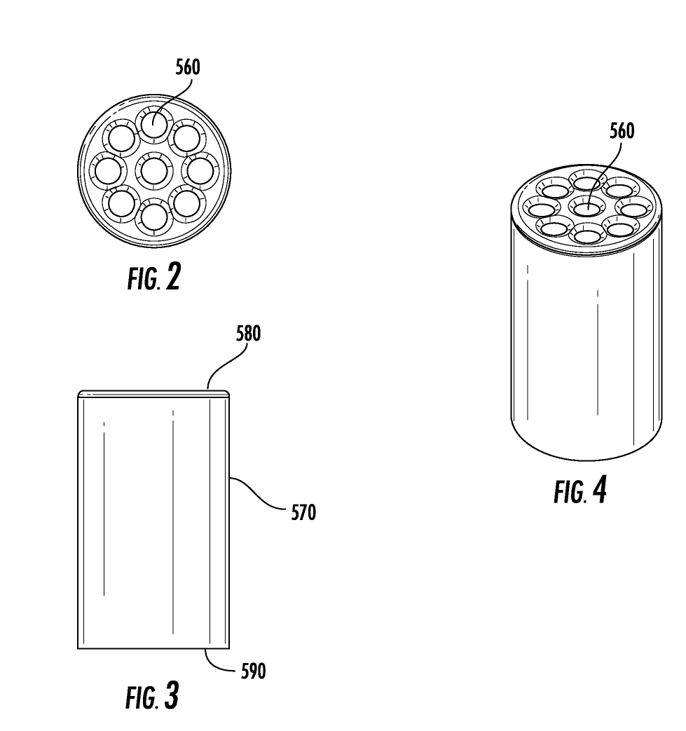

FIGS. 2, 3, and 4 are views of the downstream streamlining silencer component of the self-contained continuous positive airway pressure mask in accordance with embodiments of the disclosure. The hole diameter and taper 560. The diameter of the holes 560 can vary 0.5 mm to 7 mm and can vary with different combinations of hole sizes. The taper can go from 0-90° but can also be a combination of different taper angles. The taper is the hole diameter change angle 560. The height of the silencer/tubes 570 can vary from 1 mm to 10 cm. Increasing the length will result in increased noise reduction. This is because the air has a longer path to follow which will result in an increased streamline affect. The longer the distance the air has to travel also reduces the distance the pressure waves travel created from the intake, which reduces the noise caused by the intake.

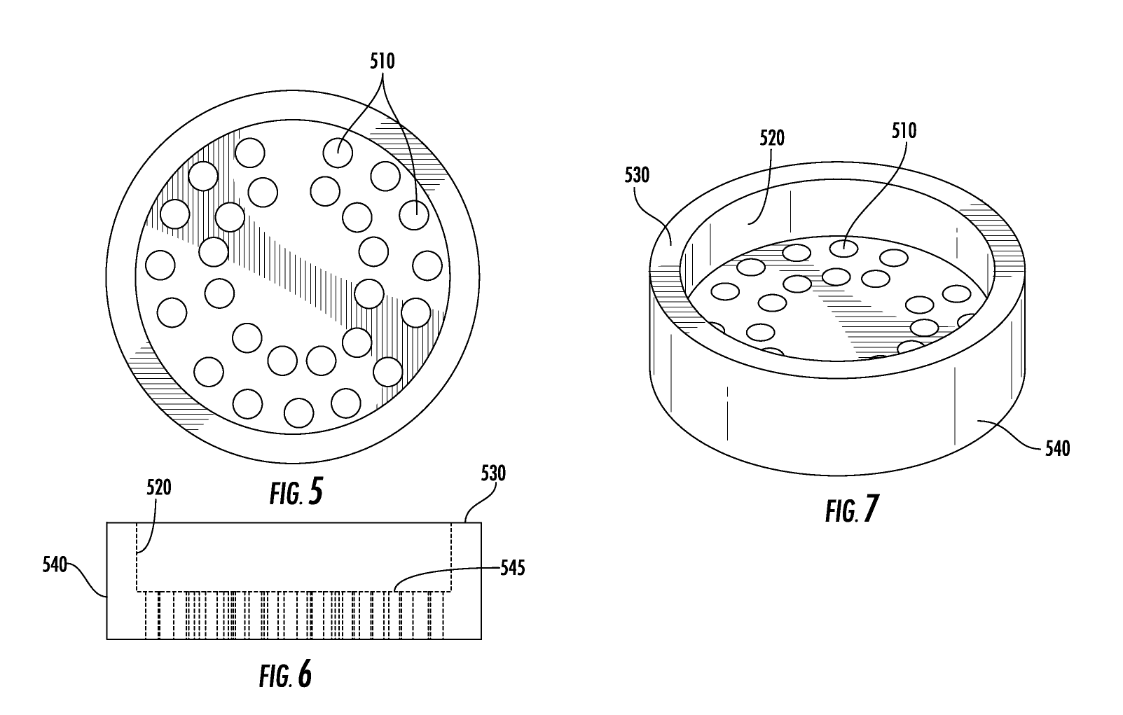

FIGS. 5, 6, and 7 are views of the upstream streamlining silencer component of the self-contained continuous positive airway pressure mask in accordance with embodiments of the disclosure. The hole diameter and taper 510 (the taper is located on the opposite side shown). The diameter of the holes 510 can vary 0.5 mm to 7 mm and can vary with different combinations of hole sizes. The taper can go from 0-90° but can also be a combination of different taper angles. The height of the silencer/tubes 540 may vary from 1 mm to 10 cm.

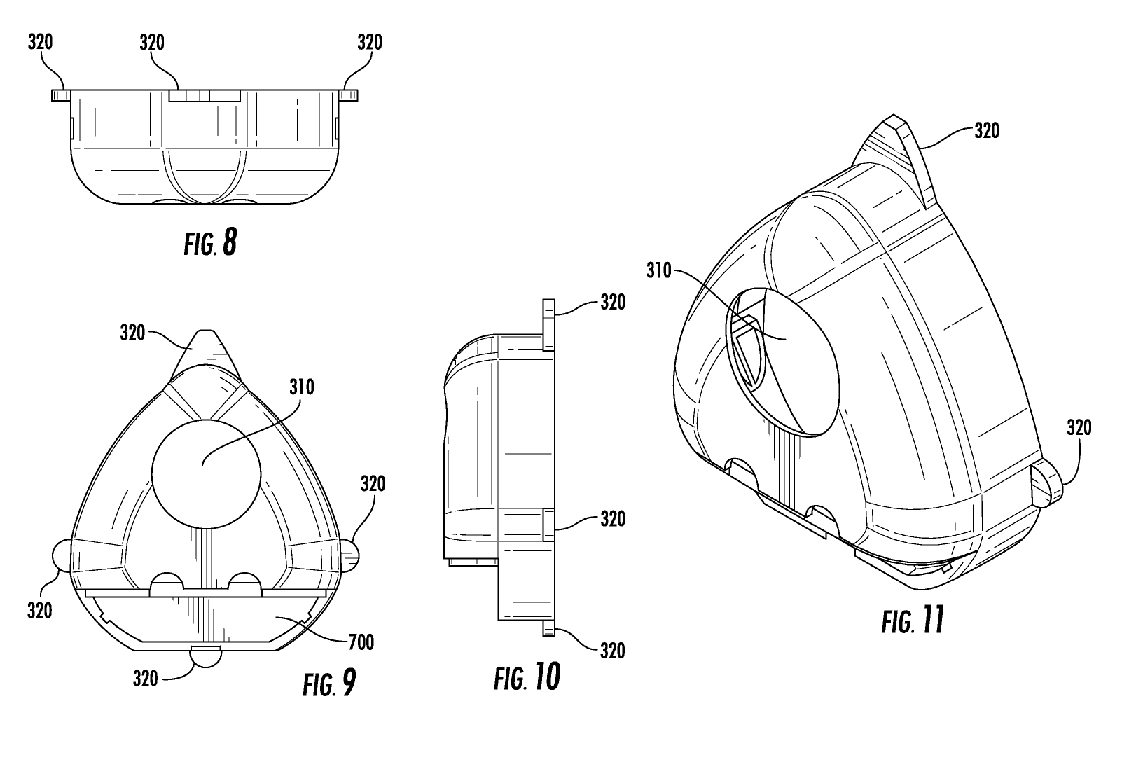

FIGS. 8, 9, 10 and 11 are views of the blower cover component 300 of the self-contained continuous positive airway pressure mask in accordance with embodiments of the disclosure. In this example the blower cover component 300 does not hold the air filter. In another instance the blower cover component 300 may hold the air filter with an attaching mechanism such as threads, clips, or any other vice used to attach the filter. In another instance the blower cover component 300 has the silencer/streamlining apparatus built into the actual cover as one component. In another instance the blower cover component 300 contains an apparatus for attaching the silencing mechanisms. The blower cover component 300 may also include a mechanism for holding the humidity wick 700. The blower cover component 300 may also include a mechanism used to diffuse/mystify water into the airstream for supplied humidity. In another embodiment blower cover component 300 contains a mechanism used to diffuse/mystify air in the humidity wick to maintain humidity supplied to the patient. The blower cover component 300 in an embodiment includes attachment means, where in an embodiment they are tabs 320 with holes to mount the blower cover component 300 to the mask base 100 with screws. The blower cover component 300 may not include holes that allow for a screw mount to the mask base 100 but instead use a clipping mechanism to where the blower cover component 300 clips to the mask base component 100 to allowing for quick detachment/attachment. In embodiments, the blower cover components is further comprised of an opening portion 310 for receiving or allowing air to enter the mask.

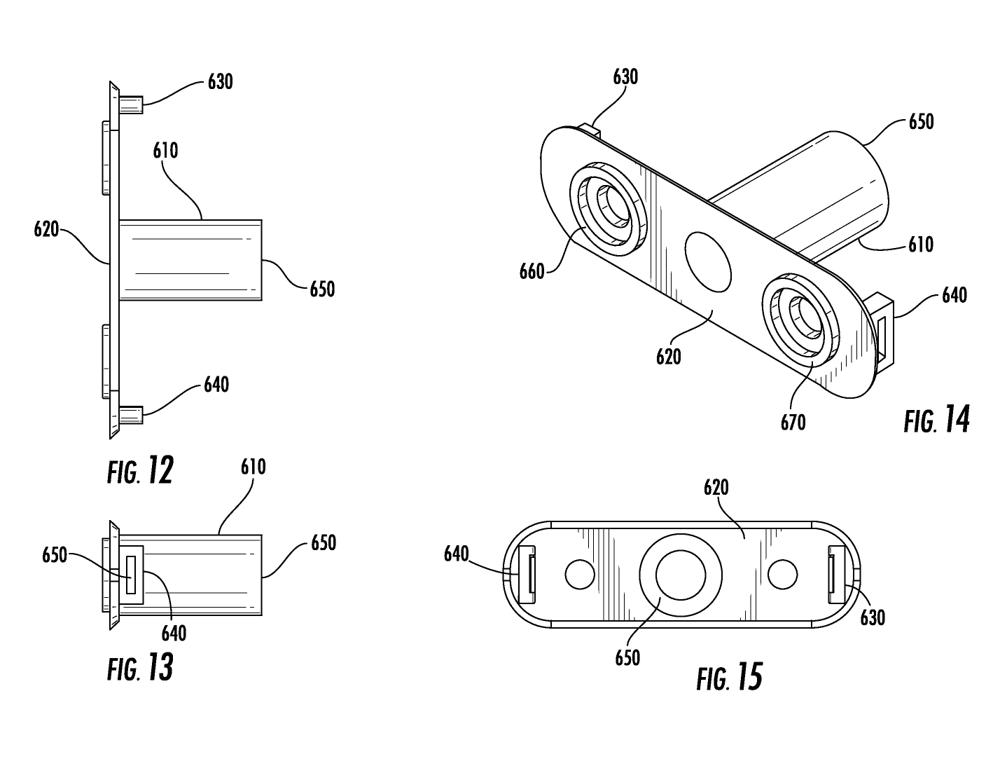

FIGS. 12, 13, 14, and 15 are views of the forehead assembly component 600 of the self-contained continuous positive airway pressure mask in accordance with embodiments of the disclosure. This component may be threaded, clipped, pinned, spring loaded, or consist of multiple components used to adjust the mask to the wearer's face and alleviate weight felt on the wearer's nose. In embodiments, the forehead support component 600 contains attachment holes 660, 670 to allow for prefabricated off the shelf rubber padding to be inserted. In embodiments, the forehead support component 600 also includes two slots 630, 640 that allow for straps to be run through allowing for the mask to be mounted to the wearer's face. The end portion 650 may be tubular 610 and inserted into the mask base component 100 as a means of attachment to the mask base component.

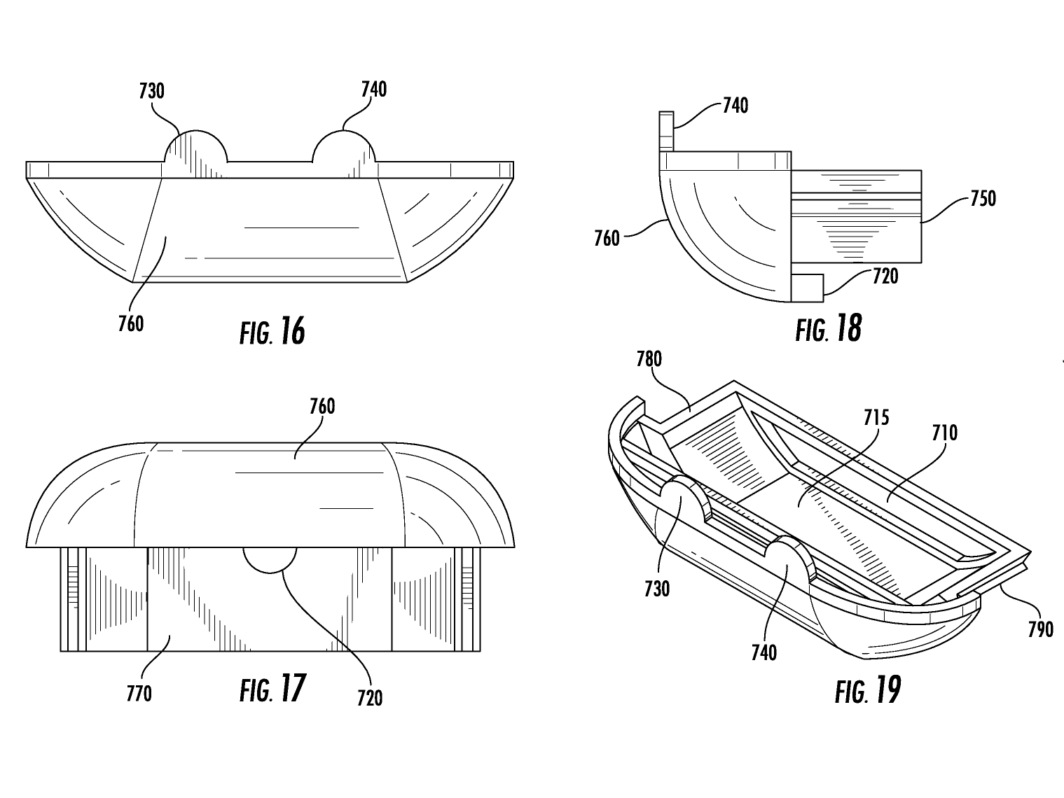

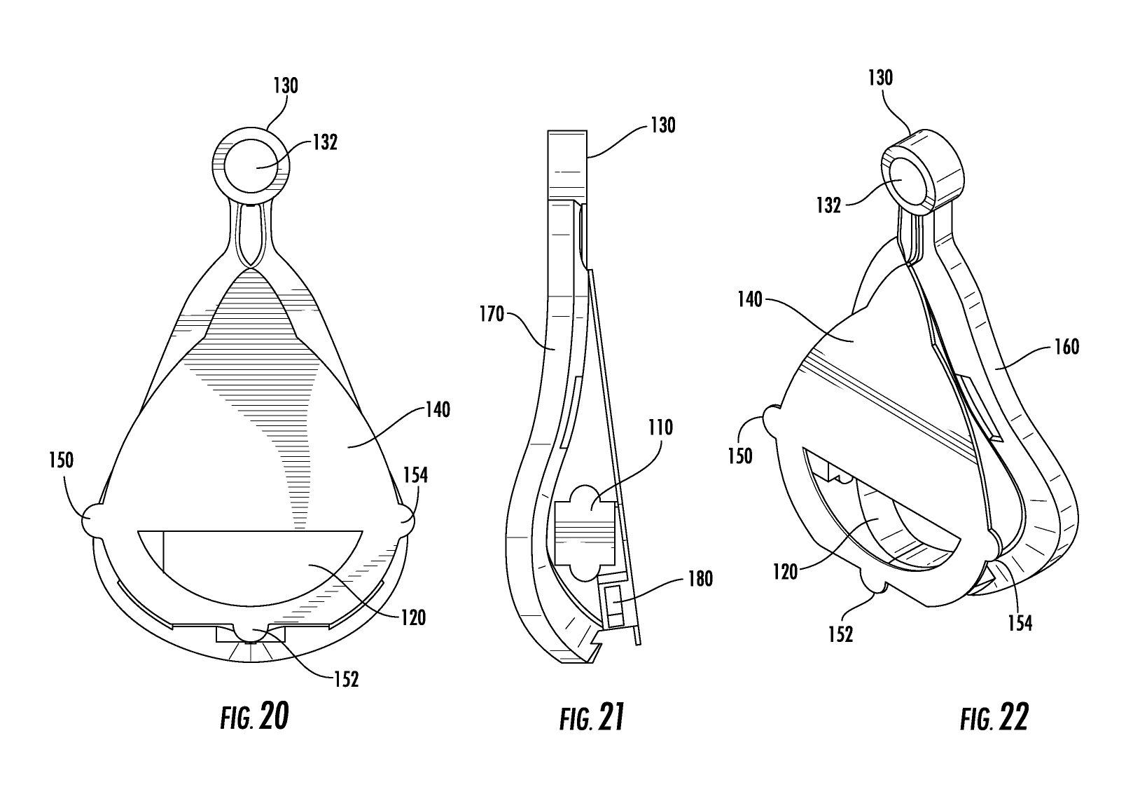

FIGS. 16, 17, 18, and 19 are views of the humidification tray component 700 of the self-contained continuous positive airway pressure mask in accordance with embodiments of the disclosure. The tray 700 uses a sliding mechanism consistent with the blower cover component 300. The dimensions of the humidification tray component 300 are tailored to the dimension of the blower cover component 300. The humidification tray component 700 holds the pre-saturated humidity wick for pass-over humidity. The humidification tray component 700 may also include a port for which water can be added to the wick via piezoelectric pump and water pouch. In another embodiment the humidification tray component 700 may consist of a hinged mechanism that pivots open to allow for easy access to the humidification wick. Knobs or protrusion areas 720, 730, 740 are used in embodiments to assist the humidification tray component 700 to slide and stop flush with the blower cover component 300. The humidification tray 700 in an embodiment has an opening portion 710 that is opens into the internal area of the mask base component 100 or blower cover component 300.

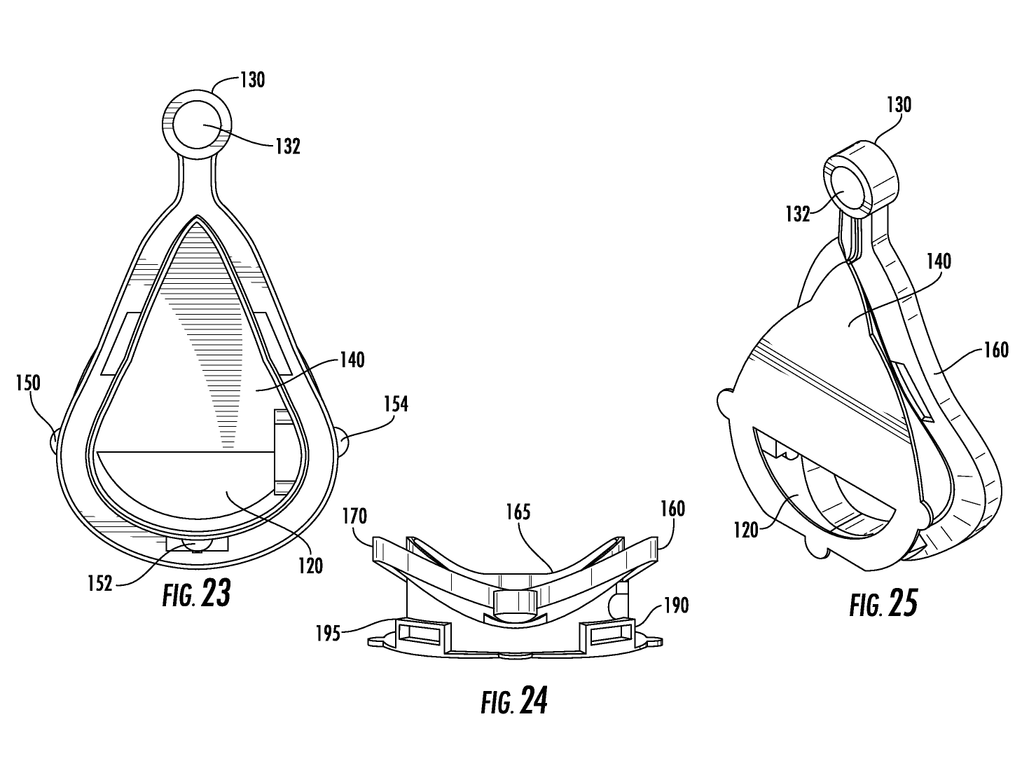

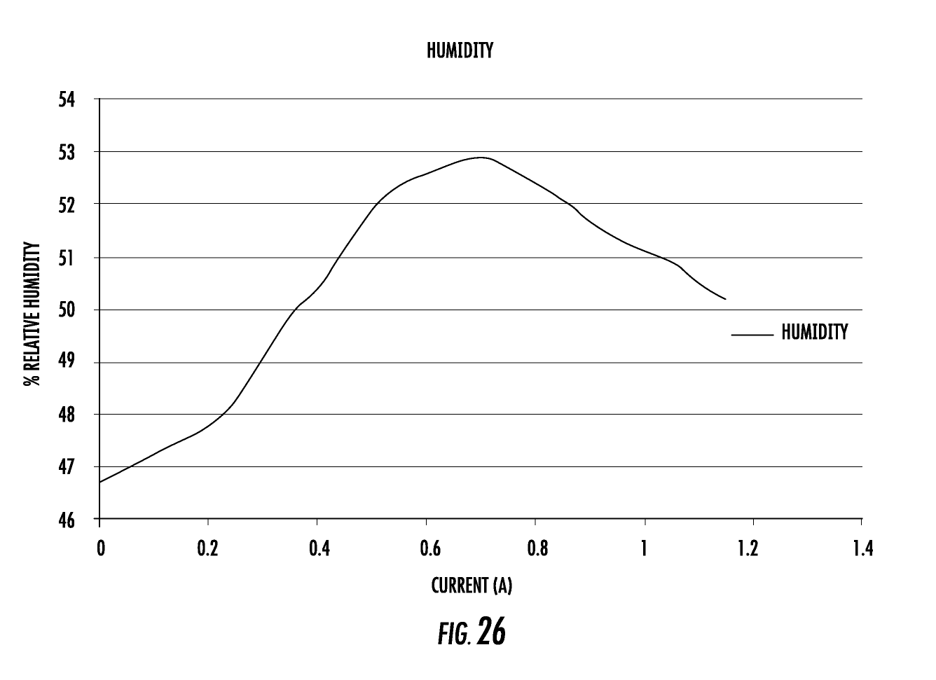

FIGS. 20, 21, 22, 23, 24 and 25 are views of the mask base component 100 of the self-contained continuous positive airway pressure mask in accordance with embodiments of the disclosure. The mask base component 100 includes an attachment portion 130, 132 at the top of the mask base component 100 for a forehead support. In another embodiment the attachment portion 130, 132 may not be included whereas other mechanisms may be used to divert the weight from the nose to another location such as the cheeks of the wearer's face. In embodiments, the mask base component 100 contains a slot or opening portion 110 to hold the pressure/temperature sensor 400 allowing for measurements in temperature and pressure to be taken on the inside of the mask. The mask base component 100 in embodiments includes an outer siding or slots 160, 170 to allow for prefabricated off the shelf face mask seals 165 to be inserted. The mask base component 100 includes a main opening portion 120 to allow for the flow of air from the blower component 200 to be pushed through the mask base component 100 and to the wearer. The mask base component 100 may also include a mechanism to allow for water to be diffused/mystified into the airstream by a separate hose, piezoelectric pump, and water pouch. In embodiments, the mask base component 100 is further comprised of attachment holes 190, 195 to allow straps or other attachment means to attach the mask to the wearer.

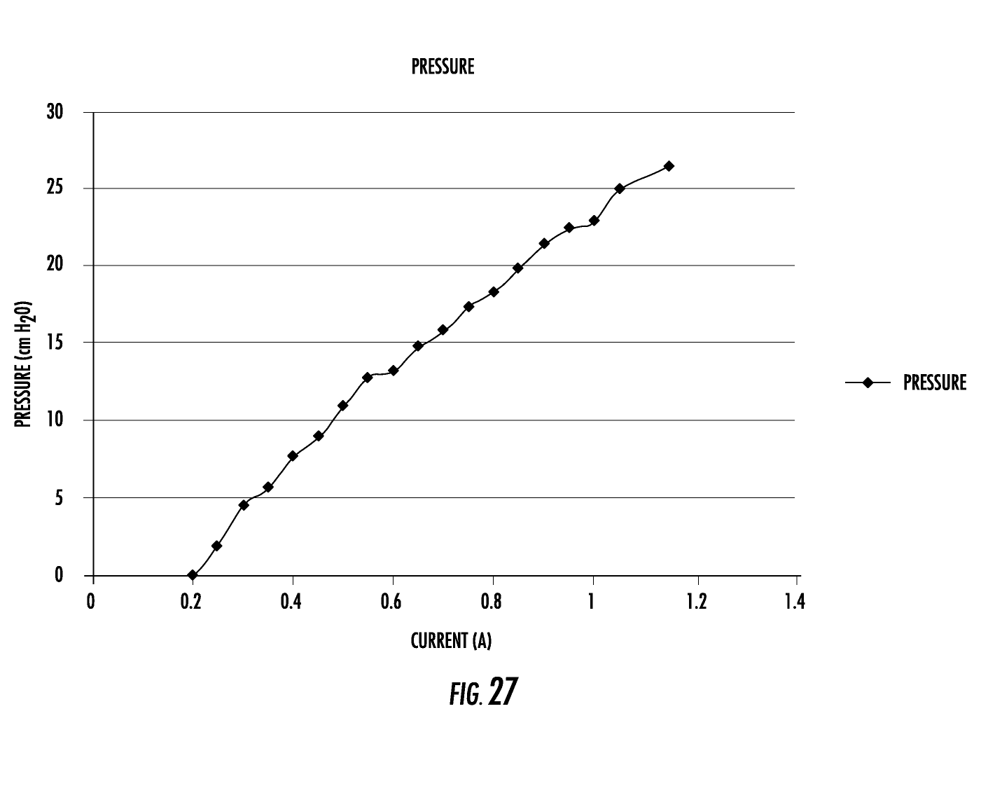

Next, reference is made to FIG. 26 and FIG. 27. FIG. 26 is a result set plot of relative humidity versus current of the self-contained continuous positive airway pressure mask in accordance with embodiments of the disclosure. FIG. 27 is a result set plot of pressure versus current of the self-contained continuous positive airway pressure mask in accordance with embodiments of the disclosure.

Reference is now made to FIG. 28, an exploded view of the self-contained continuous positive airway pressure mask in accordance with embodiments of the disclosure. Shown in this illustration are the control system components added. In an embodiment the control system component is comprised of at least one pressure sensor 400. In another embodiment, the control system component is further comprised of at least one humidity sensor 405. In a further embodiment, the control system components is further configured with at least one control board 1300, 1400, and a voltage regulator 1200 for the controllers 1300, 1400 and sensors 400, 405. In an embodiment, a proportional integral derivative (PID) control board 1300 is used to control (speed of the blower component) the blower control board 1400. In an embodiment, an LCD interface 1100 is provided to the wearer of the mask to control or provide inputs to the mask. In other embodiments, an intake component 1500 is configured into the mask which is aided in some embodiments by an addition tube or funnel portion 1600. The intake component 1500 funnels the air flow from the blower component 200 to the base mask component 100 at the opening portion 120. A power source connector 1700 is also configured in some embodiments. In yet another embodiment, a diffuser component 900 is configured as part of the mask. The aim of the diffuser component 900 is to redirect the flow of air down and away from the wearer's face. That is, instead of the air flow blowing directly towards the wearer's face, the air or gas flow will be directed down. Also shown in this illustration is in an embodiment, an additional silencing cover component 1000 is used to further reduce the noise of the mask during operation. The silencing cover component having an air flow opening 1010 and attachment portions 1020, 1030.

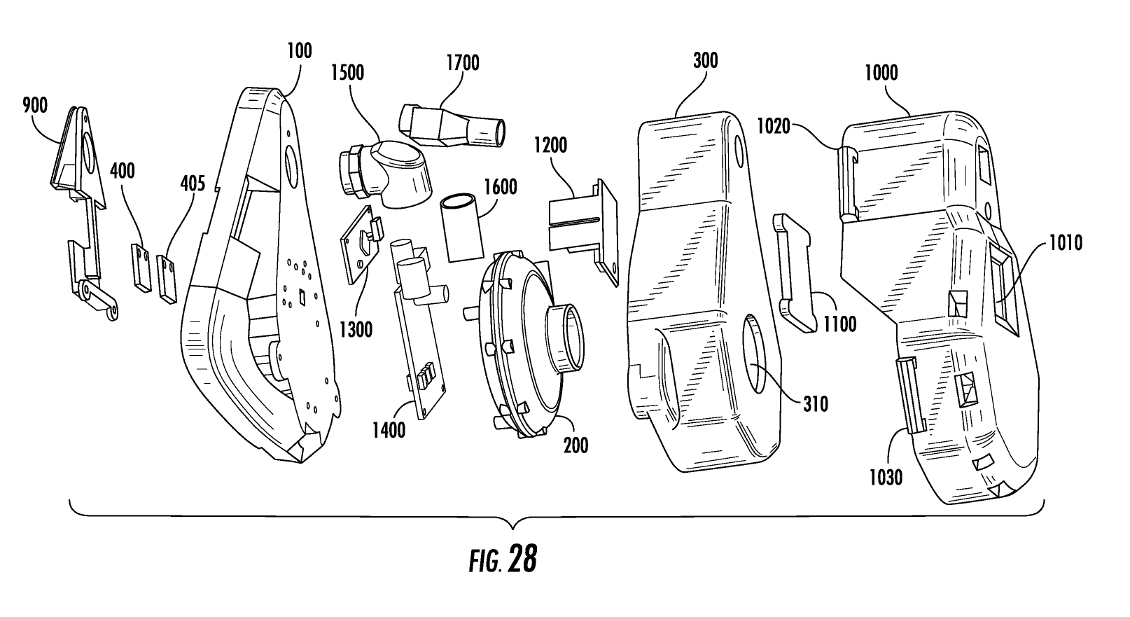

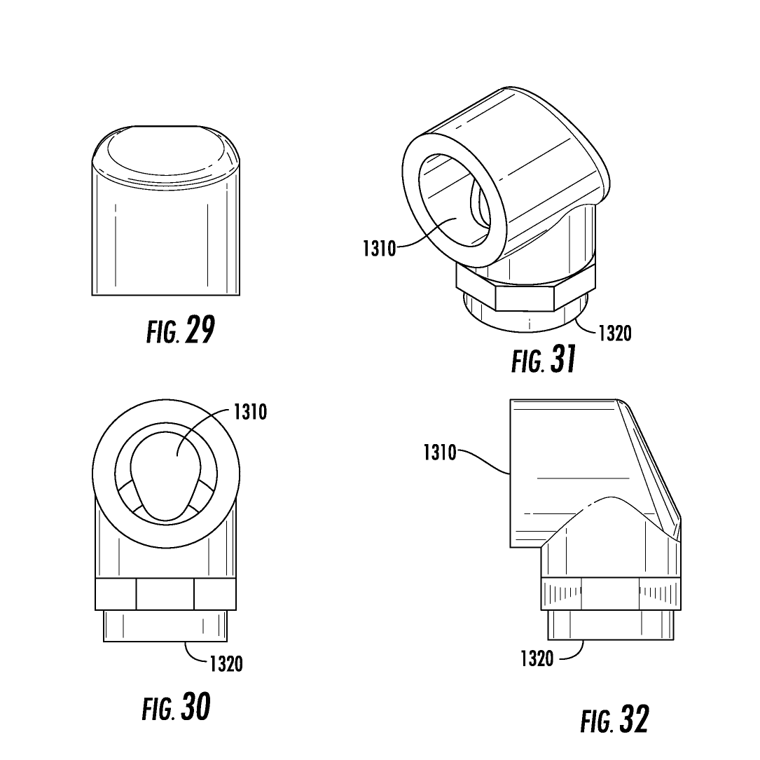

Reference is now made to FIGS. 29, 30, 31, and 32, views of the intake component 1500 of the self-contained continuous positive airway pressure mask in accordance with embodiments of the disclosure. Shown here is the tubular structure of the component. The function of this component is to direct the air flow from the blower component 200 to the mask base component 100 opening portion 120. The airflow enters the component from one end 1310 to the other 1320.

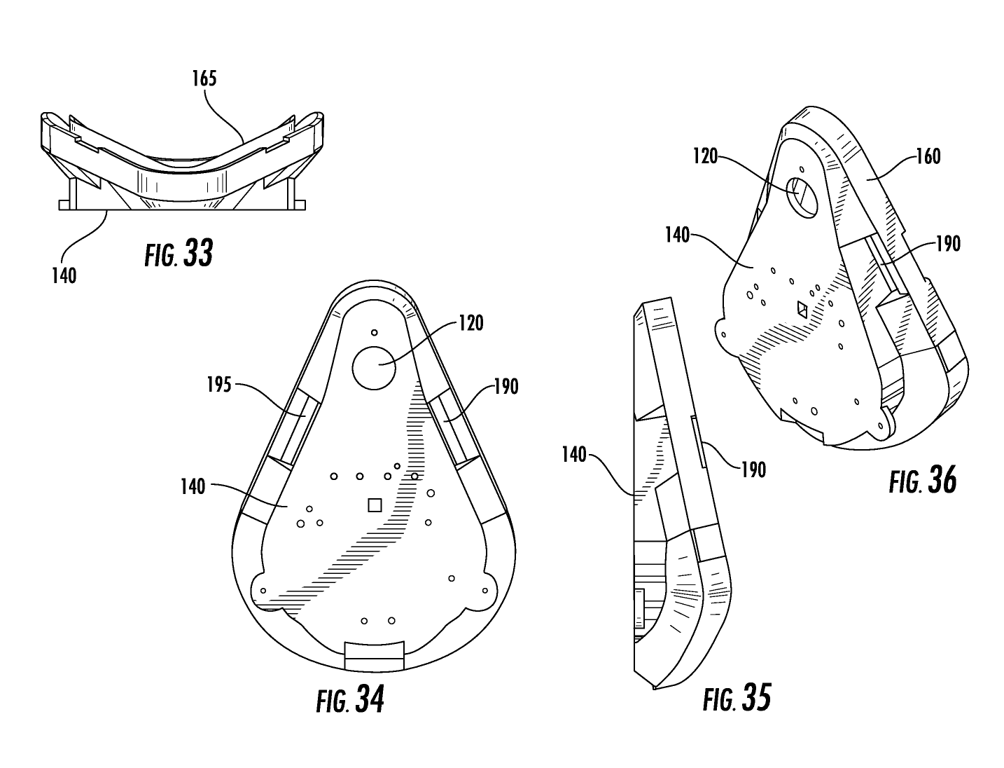

Reference is next made to FIGS. 33, 34, 35, and 36, views of the mask base component 100 of the self-contained continuous positive airway pressure mask in accordance with embodiments of the disclosure. The face portion 140 of the mask base component 100 is shown which allows in embodiments, the other components (control system component, blower component, and blower cover component) to be mounted. Attachment portions are also shown 190, 195 as well as the air flow opening portion 120. The face mask seals 165 are also shown attached.

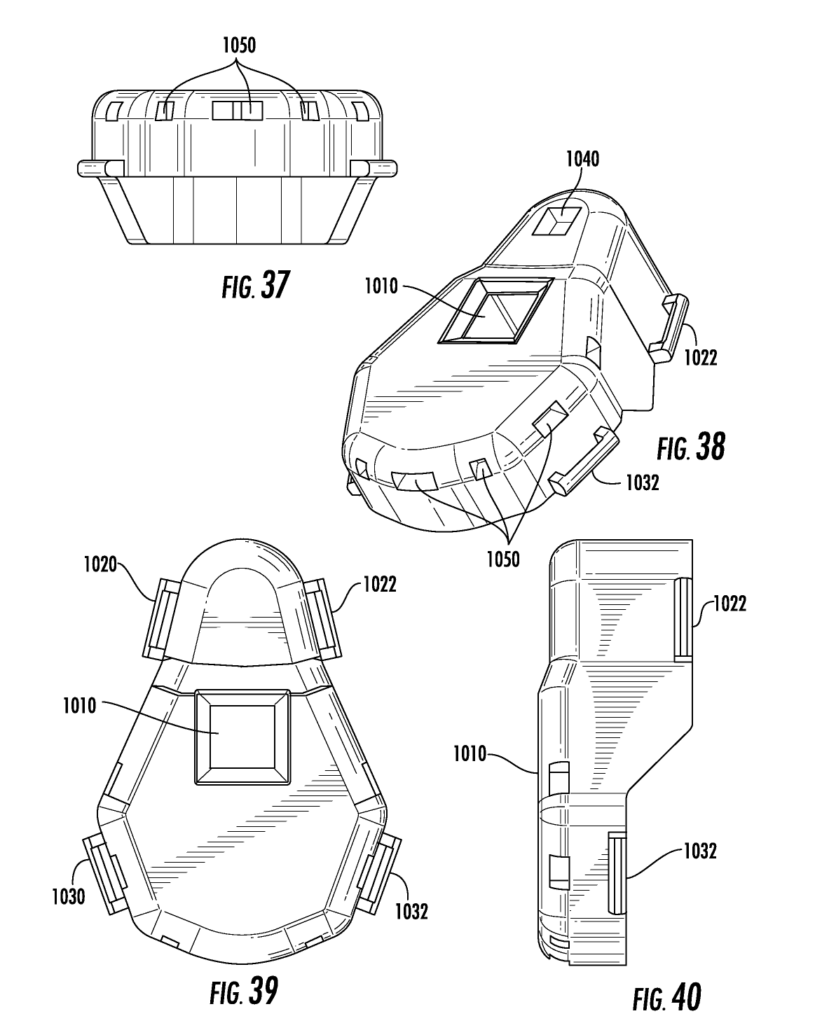

Reference is now made to FIGS. 37, 38, 39, and 40 a bottom view of the silencing cover component 1000 of the self-contained continuous positive airway pressure mask in accordance with embodiments of the disclosure. This component used to further reduce the noise of the mask during operation. The silencing cover component in embodiments has an air flow opening 1010 for the gas/air to enter and be funneled to the wearer as well as attachment portions 1020, 1022, 1030, 1032. Additional openings 1050 may be introduced to assist in the ventilation and release of expended gas/air.

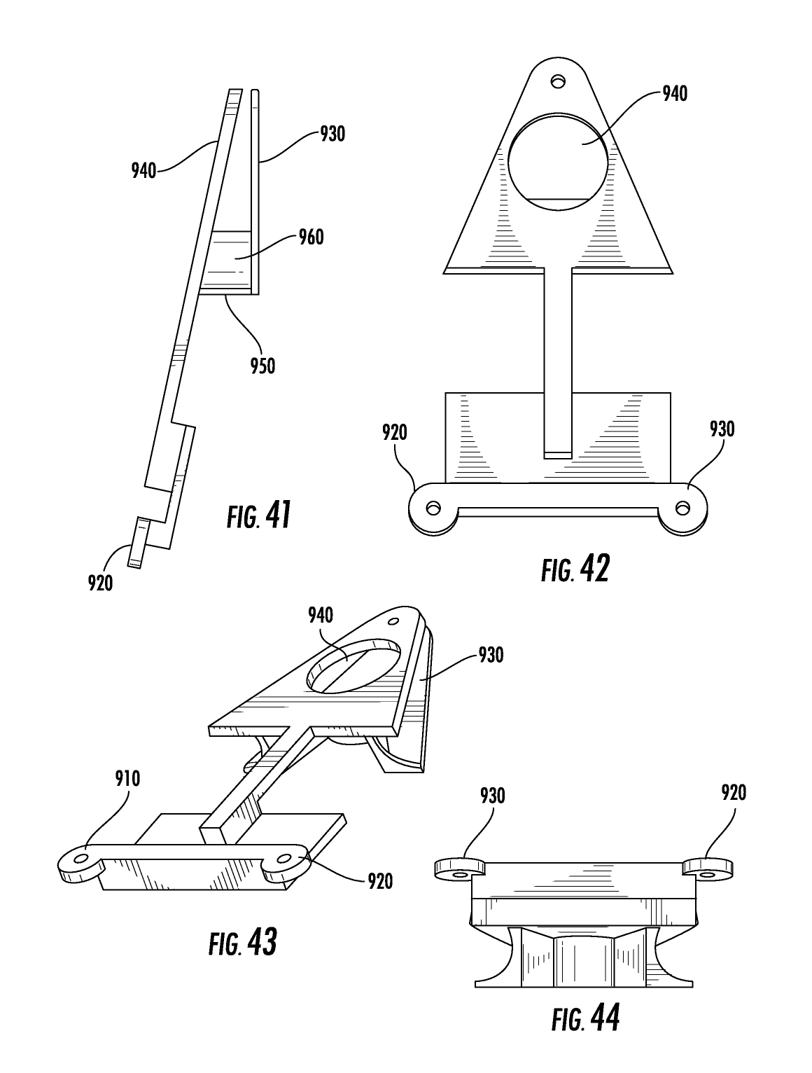

Reference is next made to FIGS. 41, 42, 43, and 44 a side view of the diffuser component 900 of the self-contained continuous positive airway pressure mask in accordance with embodiments of the disclosure. The diffuser component 900 redirects the flow of air down and away from the wearer's face. That is, instead of the air flow blowing directly towards the wearer's face, the air or gas flow will be directed down. This component is mounted to the mask base component's 100 back side and funnels the air from the opening portion 940 against the back plate portion 930 which diffuses or redirects the flow of air down through the bottom 950 of the back plate portion 930. The diffuser component 900 may also be comprised in embodiment of attachment portions 910, 920, 930 used to screw or mount the component to the mask base component 100.

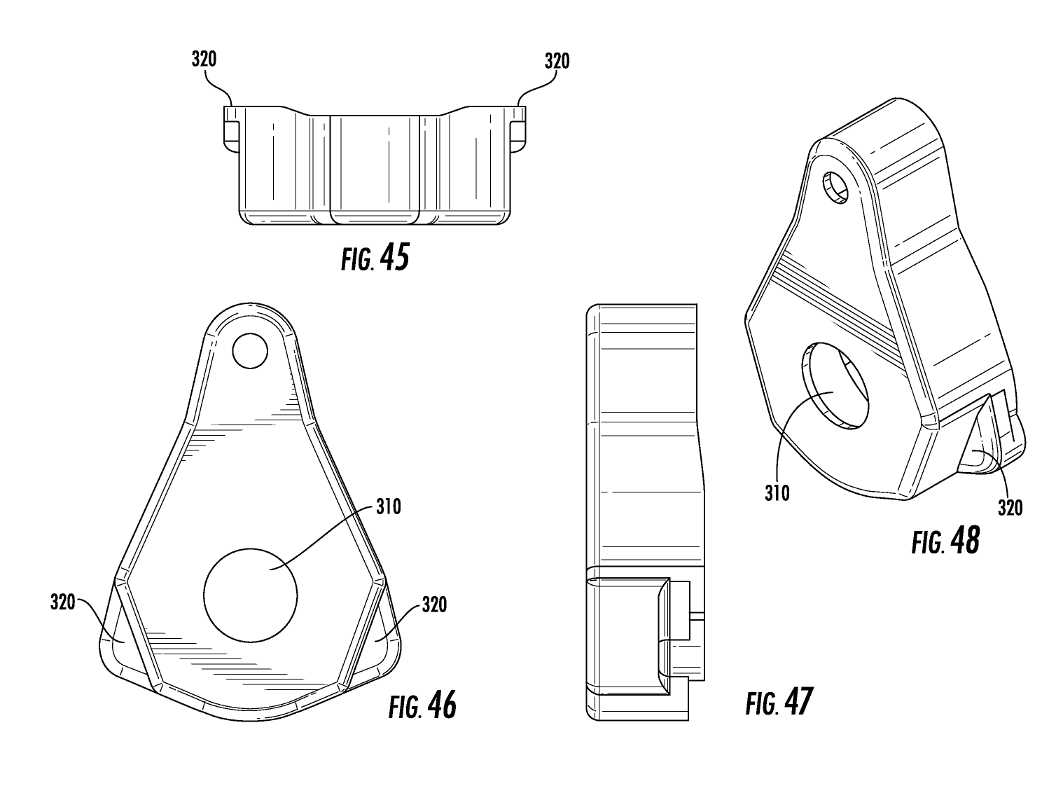

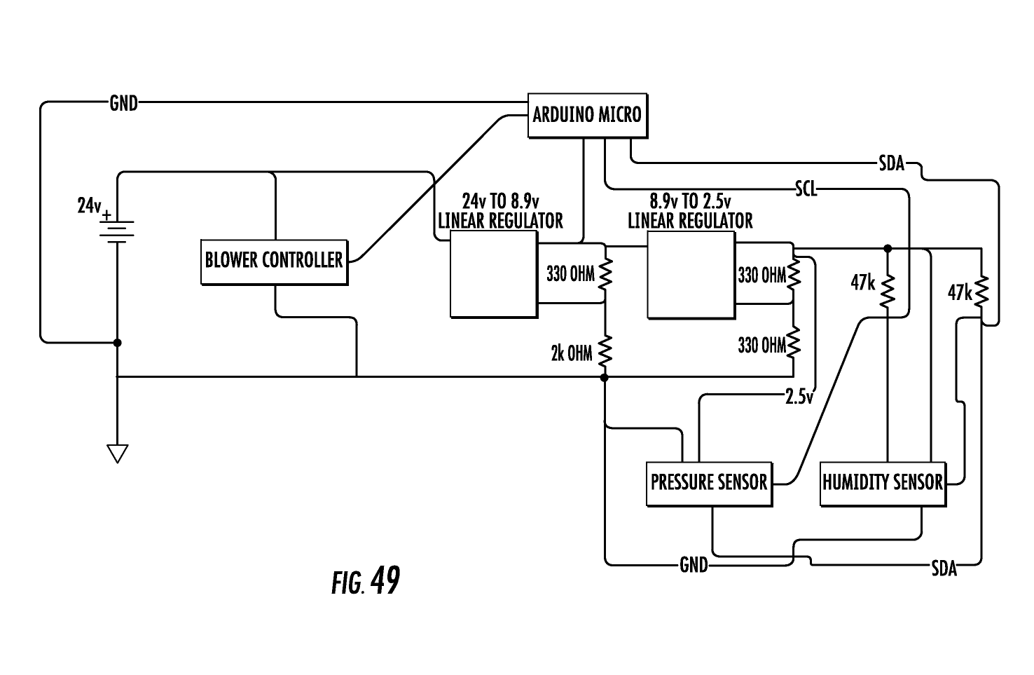

Reference is now made to FIGS. 45, 46, 47, and 48 a top view of the blower cover component 300 of the self-contained continuous positive airway pressure mask in accordance with embodiments of the disclosure. Illustrated here is another rendering of the blower cover component 300.

Reference is lastly made to FIG. 49, a wiring schematic of the self-contained continuous positive airway pressure mask in accordance with embodiments of the disclosure. Illustrated here is how the control system components are connected. In an embodiment, the control system is configured to detect when the wearer is having an apnea episode and only applies positive pressure for the duration of the apnea episode.

In brief, as described herein provides for an effective and efficient self-contained continuous positive airway pressure mask.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention.

Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Any embodiments comprising a one component or a multi-component device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications, references, patents, and patent applications mentioned in the specification are indicative of the level of those skilled in the art to which this invention pertains. All publications, references, patents, and patent application are herein incorporated by reference to the same extent as if each individual publication, reference, patent, or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The devices and/or methods disclosed and claimed herein can be made and executed without undue experimentation in light of the present disclosure. While the device and methods of this invention have been described in terms of preferred embodiments, it will be apparent to those skilled in the art that variations may be applied to the device and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A self-contained continuous positive airway pressure mask comprising:
a mask base component configured to be placed and secured to a wearer's face thereby forming a chamber between said base component and the wearer's face with an opening portion for receiving breathable gas and an outlet portion for relieving gas;
a blower component mounted to said mask base component and being configured to create a pressure in said chamber;
a blower cover component mounted to said mask base component and configured to cover the blower component; and
a control system component for maintaining said pressure in said chamber.

2. The mask of claim 1, wherein said blower component is configured to create a pressure of about 2-20 cm H2O in said chamber.

3. The mask of claim 1, wherein said mask base component is configured to cover the wearer's nasal region.

4. The mask of claim 1, wherein said mask base component is configured to cover the wearer's oral region.

5. The mask of claim 1, wherein said mask base component is configured to cover the wearer's nasal and oral region.

6. The mask of claim 1, wherein said control system component further comprises a pressure sensor.

7. The mask of claim 1, wherein said control system component further comprises a humidity sensor.

8. The mask of claim 1, wherein said control system further comprises a pressure sensor, a humidity sensor, at least one control board, a voltage regulator for said sensors and said control board.

9. The mask of claim 8, wherein at least one control board is a PID control board.

10. The mask of claim 1, further configured with a power source.

11. The mask of claim 10, wherein said power source is mounted within said blower cover.

12. The mask of claim 10, wherein said power source is a battery source.

13. The mask of claim 1, further configured to allow the connection to a power source.

14. The mask of claim 1, further configured with a downstream silencer component.

15. The mask of claim 1, further configured with an upstream silencer component.

16. The mask of claim 1, further configured with a forehead assembly component.

17. The mask of claim 1, further configured with a humidification component.

18. The mask of claim 1, further configured with a silencing component.

19. The mask of claim 1, further configured with an intake component.

20. The mask of claim 1, further configured with a diffuser component.
